(19) United States
(12) Patent Application Publication
(10) Pub. No.: US 2002/0198436 A1
(43) Pub. Date: Dec. 26, 2002

(54) Circulation assist method and device utilizing balloon of IABP system and blood stream control valve therefor

(76) Inventor: Motoaki Hoshino, Tenpaku-ku (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/942,656

(22) Filed: Aug. 31, 2001

(30) Foreign Application Priority Data
Jun. 21, 2001 (JP) 2001-188580

(51) Int. Cl. A61M 1/12

(52) U.S. Cl. 600/18

(57) ABSTRACT

A circulation assist method and device is provided wherein the contraction and expansion of a balloon of an IABP system which has already been widespread as a device for pressure support are utilized as power source for operating a pump so as to realize the assist of stream volume. A cylindrical pump chamber is connected at one and the other ends thereof respectively with an inflow hole and an outflow hole. An inflow valve is provided at the side of the inflow hole for permitting blood to stream from the inflow hole into the pump chamber but preventing the backward stream, and an outflow valve is further provided at the side of the outflow hole for permitting blood to stream from the pump chamber into the outflow hole but preventing the backward stream. A balloon portion of a balloon catheter connected to the IABP system is fluid-tightly incorporated into the pump chamber and is pulsated, so that blood is sucked into the pump chamber through the inflow valve when the balloon is contracted, and is ejected therefrom through the outflow valve when the balloon is expanded.

### BACKGROUND OF THE INVENTION

1. Field of the Invention:

The present invention relates to a circulation assist method and device and a blood stream control valve for mechanically replacing or assisting the pumping function of a heart for a short period of time for the purpose of treatment for serious heart failure.

2. Discussion of the Prior Art:

As assisted circulation, there are known mechanical assist means such as IABP, LVAD, V-A Pumping (PCPS by means of an artificial cardiopulmonary or a centrifugal pump) and the like. Of these, the IABP has been widespread these days. It is to be noted that the abbreviations used above means the following definitions herein:

IABP: Intra-Aortic Balloon Pumping

LVAD: Left Ventricular Assist Device

V-A: Veno-Arterial

PCPS: Perctaneous Cardiopulmonary Support

In the IABP, a cannula is inserted from the thigh artery to set a balloon in the thoracic descending aorta. Gas is ejected into and sucked from the balloon, whereby the same is alternately expanded and contracted thereby to reverse the phase of blood pressure. Namely, the IABP is an assist means which aims at performing systolic unloading and disastolic augmentation and has been generally used for the reason that it can be easily manipulated even by physicians. On the other hand, the LVAD is an assist means for bypassing the left ventricle directly from the suction cannula of the left atrium by way of an artificial heart pump. This has been experienced only at a few facilities even in our country because the drive unit and the artificial heart pump are expensive as well. Centrifugal pumps are comparatively low price and easy to use and recently, are coming into wide use. However, the V-A pumping has many difficulties such as, for example, the incorporation of an artificial lung into the human blood circuit.

Further, although the IABP has been widespread as a circulation assist device which is useful for acute myocardial infarction and acute heart failure subsequent to cardiac surgery operation, it has a problem in that the effect is pressure assist but not stream assist. For more serious heart failure which needs assisting the volume of stream, it is often the case that there is required an upper, powerful circulation assist means which uses such a pump as LVAD or PCPS.

### SUMMARY OF THE INVENTION

Accordingly, it is a primary object of the present invention to provide a circulation assist method and device capable of assisting the volume of blood stream in cooperation with the widespread IABP system during a heart surgery operation.

Another object of the present invention is to provide a circulation assist method and device of the character set forth above which is easy to handle, simple in construction and low-priced.

A further object of the present invention is to provide a circulation assist method and device which utilizes the expansion and contraction motion of a balloon of the widespread IABP system in making one-way stream of blood, in other words, which realizes assisting the volume of stream under the use of a pressure assist device by converting the motion of a balloon of the pressure assist device into the power source for pumping.

An additional object of the present invention is to provide a blood stream control valve capable of streaming blood in a one-way direction, which valve is particularly designed for use in the circulation assist method according to the present invention.

Briefly, according to the present invention, there is provided a circulation assist method which utilizes a balloon of an IABP system. The method comprises the steps of: providing a generally cylindrical pump chamber whose one and the other ends respectively communicate with an inflow hole and an outflow hole; providing an inflow valve for permitting blood to stream from the inflow hole into the pump chamber but for preventing the backward stream of blood; providing an outflow valve for permitting blood to stream from the pump chamber to the outflow hole but for preventing the backward stream of blood; connecting the inflow hole to a suction cannula which is inserted into and secured to the left atrium of a patient's heart; connecting the outflow hole to an ejection cannula which is inserted into and secured to the patient's aorta, thereby bypassing the left ventricle; fluid-tightly incorporating into the pump chamber a balloon portion of a balloon catheter of an IABP drive unit; connecting the balloon catheter to the IABP drive unit; and contracting and expanding the balloon with the timings determined based on trigger signals which a trigger circuit of the IABP drive unit selects from living body signals of the patient, so as to obtain one-way stream of blood.

With this configuration, by using the pump chamber for incorporating therein the balloon of the balloon catheter and the inflow valve and the outflow valve in combination with the widespread IABP system, it can be realized to bypass the left ventricle with the blood stream being secured at a desired pressure and a required volume.

In another aspect of the present invention, a cylindrical pump chamber is provided with one and the other ends thereof connected respectively to an inflow hole and an outflow hole. An inflow valve is provided for permitting blood to stream from the inflow hole into the pump chamber but for preventing the backward stream of blood, and an outflow valve is further provided for permitting blood to stream from the pump chamber to the outflow hole but for preventing the backward stream of blood. A balloon portion of a balloon catheter connected to an IABP drive unit is fluid-tightly incorporated into the pump chamber and is pulsated. The balloon in the pump chamber of the balloon catheter, when contracted, makes blood stream from the inflow hole into the pump chamber through the inflow valve and when expanded, makes blood stream out of the outflow hole through the outflow valve. Thus, by adding to the widespread IABR system, the pump chamber for incorporating the balloon of the balloon catheter and the inflow and outflow valves, it can be realized to provide a circulation assist device which is capable of assisting the stream of blood at a desired pressure as well as a required volume.

In still another aspect of the invention, the pump chamber is provided at a straight portion of a J-shape cylindrical member and the inflow and outflow holes are provided respectively at the opposite ends of the cylindrical member. The balloon of the balloon catheter is fluid-tightly inserted from a bent portion of the J-shape cylindrical member into the pump chamber. Thus, only by adding to the widespread IABP system the J-shape cylindrical member provided with inflow and outflow valves, it can be realized to assist the stream of blood of a required volume at a desired pressure in the state that the blood stream is smooth and that there occur little hemolysis and little thrombus.

In a further aspect of the present invention, the inner diameter of the pump chamber for containing the balloon is made the same to or slightly larger than the diameter of the balloon being expanded, and the length of the pump chamber is made as close to that of the balloon as possible so as to exclude any useless space as much as possible. Thus, in addition to the effects of claim 1 or 2, it becomes possible to reduce the volume of blood dwelling in the pump chamber to the minimum, whereby thrombosis can be prevented. Moreover, the pump chamber particularly designed to fit the size of the balloon can advantageously prevent the balloon which pulsates therein, from jumping with the motion as well as from vibrating undesirably.

In a further aspect of the present invention, a ball valve in the inflow valve is moved along guide protrusions extending in the direction of blood stream within a valve chamber which communicates with the pump chamber at the side of the inflow hole. The ball valve prevents the backward stream from the pump chamber toward the inflow hole when seated on a valve seat formed in the valve chamber at the side of the inflow hole, and the moving end of the ball valve is confined by stop portions which are formed by raising the ends at the side of the pump chamber of the guide protrusions. Similarly, a ball valve in the outflow valve is moved along guide protrusions extending in the direction of blood stream within a valve chamber which communicates with the pump chamber at the outflow side. This ball valve prevents the backward stream from the outflow hole toward the pump chamber when seated on a valve seat formed at an outflow side of the pump chamber, and the moving end of the ball valve is confined by stop portions which are formed by raising the ends at the side of the outflow hole of the guide protrusions. With this configuration, there can be obtained inflow and outflow valves which are simple in construction, less-expensive, capable of restraining hemolysis and thrombosis to the minimum and the most suitable to a circulation assist means for assisting the stream of blood temporarily.

In a still another aspect of the present invention, there is provided a blood stream control valve wherein a ball valve is movable along guide protrusions which extend in the axial direction thereof in a valve chamber. The ball valve prevents the backward stream from an outlet hole toward an inlet hole when seated on a valve seat, while it permits the stream from the inlet hole toward the outlet hole when leaving the valve seat. By raising the ends at the side of the outlet hole of the guide protrusions, stop portions are formed for confining the movable end of the ball valve. Thus, the blood stream control valve according to the present invention is simple in construction, low-priced and is capable of reducing hemolysis and thrombosis to the minimum.

In a still further aspect of the present invention, there is provided a circulation assist device used in combination or cooperation with a widespread IABP system, comprising a generally cylindrical member which is formed with a pump chamber at its mid portion and provided with an inflow valve and an outflow valve at opposite end thereof. The cylindrical member is bent at a portion close to one end thereof to provide a hole through which a balloon of a balloon catheter connected to the IABP system is inserted fluid-tightly. The balloon is alternately contracted and expanded by a gas circuit of the IABP system. When contracted, the balloon opens the inflow valve and closes the outflow valve, while when expanded, it closes the inflow valve and opens the outflow valve. This causes blood to stream into the pump chamber when the balloon is contracted and blood to flow out from the pump chamber when the balloon is expanded. With this construction, since the pumping operation is effected using the contraction and expansion actions of the balloon of the balloon catheter, the assist device is quite simple in construction and reliable in operation since it does not require its own power source for operation.

### BRIEF DESCRIPTION OF THE ACCOMPANYING DRAWINGS

The foregoing and other objects, features and many of attendant advantages of the present invention will be readily appreciated as the same becomes better understood by reference to the following detailed description of the preferred embodiments when considered in connection with the accompanying drawings, wherein like reference numerals designate identical or corresponding parts throughout the several views, and in which:

FIG. 1 shows a sectional view of a circulation assist device utilizing a balloon of an IABP system, also illustrating the method of using the device;

FIG. 2 shows an enlarged sectional view of an inflow valve;

FIG. 3 shows an enlarged sectional view of an outflow valve,

FIG. 4 shows a cross-section along the line 4-4 in FIG. 2, and

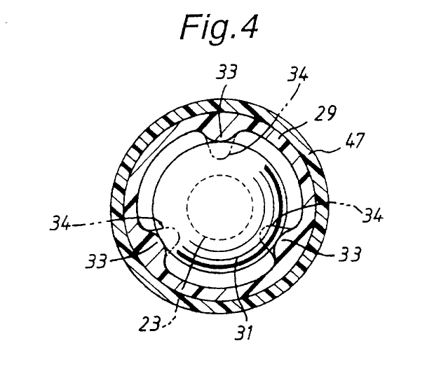

FIG. 5 shows a section of a blood stream control valve.

### DETAILED DESCRIPTION OF THE PREFERRED EMBODIMENTS

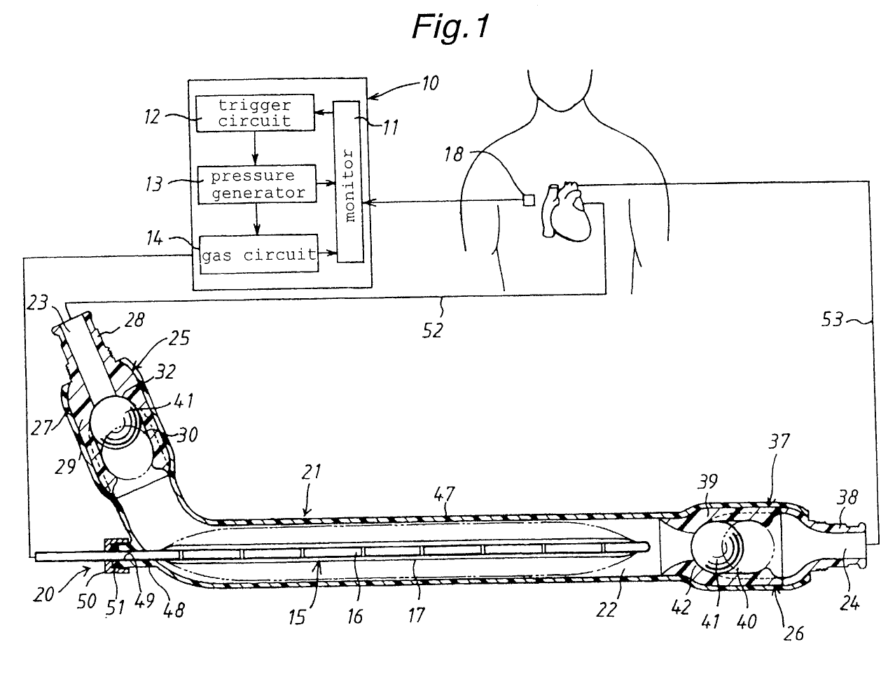

Referring now to FIG. 1, a numeral 10 denotes a drive unit for an IABP system and the unit 10 comprises a monitor 11, a trigger circuit 12, a positive/negative pressure generator 13, and gas circuit 14. The monitor 11 is connected to a detector such as an electrode 18 or the like which is affixed to a portion to be measured of a patient for taking living body signals, such as electrocardiogram, arterial blood pressure wave or the like. The trigger circuit 14 selects as trigger signals the R-wave of the electrocardiogram (electrocardiogram trigger) or the arterial blood pressure wave (arterial blood pressure trigger) and with a suitable time delay, controls the timings of the contraction and expansion of a balloon. A balloon catheter 15 connected to the IABP drive unit 10 is of the type that a slender balloon 17 is attached to an extreme end of a catheter 16.

A numeral 21 denotes a J-shape tubular or cylindrical member which constitutes a circulation assist device 20 utilizing the balloon 17 of an IABP system according to the present invention. The cylindrical member 21 is formed at its mid straight portion with a cylindrical pump chamber 22 for containing the balloon 17, and at its end close to a bent portion, with an inflow hole 23 communicating with the inflow side of the pump chamber 22. The cylindrical member 21 is further formed at the extreme end of its straight portion, with an outflow hole 24 communicating with the outlet side of the pump chamber 22. At a portion close to the inflow hole 23, there is provided an inflow valve 25 which permits blood to stream from the inflow hole 23 to the pump chamber 22 but prevents the adverse or backward stream, while at a portion close to the outflow hole 24, there is provided an outflow valve 26 which permits blood to stream from the pump chamber 22 to the outflow hole 24 but prevents the backward stream.

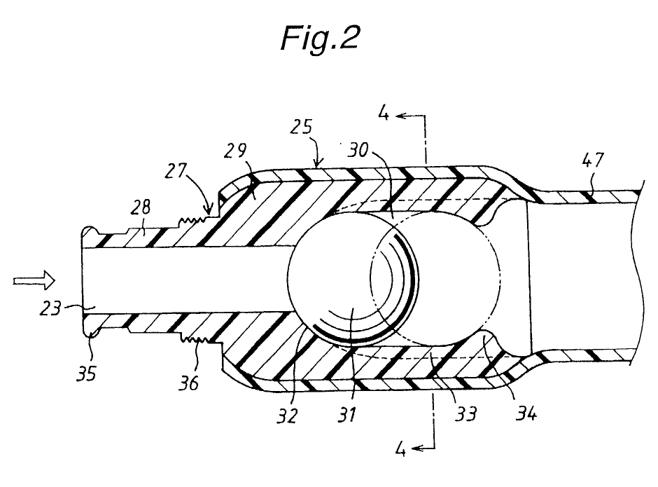

An inflow end member 27 is provided at the end of the inflow side of the J-shape cylindrical member 21 and is formed with a small diameter coupling section 28 connected to a suction cannula 52 and a large diameter valve section 29 containing an inflow valve 25. Within the coupling section 28 and coaxially with the same, the inflow end member 27 is formed with an inflow hole 23 opening at one end thereof. In the inflow end member 27, a valve chamber 30 larger in diameter than the inflow hole 23 and opening to the other end is formed coaxially within the valve section 29 and is in communication with the pump chamber 22 at the inflow hole side. A ball valve 31 contained in the valve chamber 30 is seated on a portion where the inflow hole 23 and the valve chamber 30 merge in the middle portion or the like of the inflow end member 27 so that a valve seat 32 is formed to prevent the backward stream from the pump chamber 22 toward the inflow hole 23. For smooth stream of blood, the valve chamber 30 gently increases its diameter as it goes away from the valve seat 32. At the inner surface of the valve chamber 30, as shown in FIG. 2, guide protrusions of three or four streams whose top portions are circular in cross-section extend in the direction of blood stream so as to movably guide the ball valve 31 going away from the valve seat 32 in the direction of blood stream. Each of the guide protrusions 33 has a sufficient height from the bottom or inner surface of the valve chamber 30, so that a larger cross-section area than the cross-section area of the inflow hole 23 can be secured between the ball valve 31 being away from the valve seat 23 and the bottom surface of the valve chamber 30. Each protrusion 33 radially inwardly rises at its end close to the pump chamber 22 thereby to provide a stop portion 34 which confines the end in movement of the ball valve 31 toward the pump chamber 22. Each stop portion 34 takes a streamline shape in section in the direction of blood stream, and the top of each stop portion 34 is independent of, or separated from, that of any other stop portions 34, so that the blood stream within the inflow valve 25 is smooth and that there occur little hemolysis and little thrombus.

The inflow end member 27 is made of polycarbonate resin by injection molding. The ball valve 31 made of solid silicon resin or the like is inserted into the valve chamber 30 from the side of the stop portions 34 by effecting slight elastic deformations on the stop portions 34 as well as the ball valve 31. The outer surface of the coupling section 28 is formed with an inserting portion 35 and a male thread 36 for connection and screw engagement with a suction cannula 52 which has been inserted into and secured to the left artium of the patient's heart.

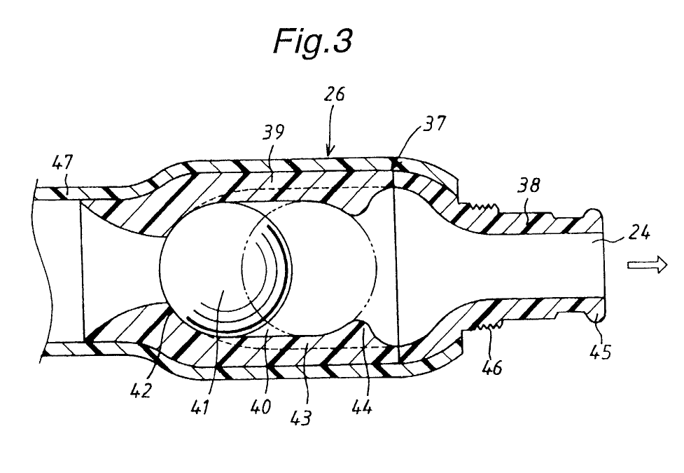

A numeral 37 denotes an outflow end member provided at the end of an outflow hole side of the J-shape cylindrical member 21. The end member 37 is formed with a small diameter coupling section 38 connected to an ejection cannula 53 and a large diameter valve section 39 containing an outflow valve 26. In the outflow end member 37, an outflow hole 24 opening at one end is formed coaxially within the coupling section 38, and a valve chamber 40 larger in diameter than the outflow hole 24 and opening at the other end is formed coaxially within the valve section 39 and is in communication with the outflow hole side of the pump chamber 22. A ball valve 41 contained in the valve chamber 40 is seated on an end close to the pump chamber 22 of the valve chamber 40, so that a valve seat 42 is formed for preventing the backward stream from the outflow hole 24 toward the pump chamber 22. For smooth stream of blood, the valve chamber 40 gently increases its diameter as it goes away from the valve seat 42. At the inner surface of the valve chamber 40, as best shown in FIG. 3, like the inflow valve 25, guide protrusions 43 of three or four streams whose top portions are semicircular in cross-section extend in the direction of blood stream so as to movably guide the ball valve 41 being away from the valve seat 42, in the direction of blood stream. Each of the guide protrusions 43 has a sufficient height from the bottom or inner surface of the valve chamber 40, so that a larger cross-section stream area than the stream area of the outflow hole 24 can be secured between the ball valve 41 being away from the valve seat 42 and the bottom surface of the valve chamber 40. Each protrusion 43 radially inwardly rises at its end close to the outflow hole 24 thereby to provide a stop portion 44 which confines the end in movement of the ball valve 41 toward the outflow hole 24. Each stop portion 44 takes a streamline shape in the direction of blood stream and the top of each stop portion 44 is independent of, or separated from, that of any other stop portions 44, so that the blood stream within the outflow valve 26 is smooth and that there occur little hemolysis and little thrombus.

The coupling section 38 and the valve section 39 of the outflow end member 37 are made separately by injection molding. The ball valve 41 made of solid silicon resin is inserted into the valve chamber 40 from the side of the stop portions 44 by effecting slight elastic deformations on the stop portions 44 and the ball valve 41. Thereafter, the sections 38 and 39 are united with each other at their end surfaces by melting joint or with a bonding agent of hot melt type. The outer surface of the coupling section 28 is formed with an inserting portion 45 and a male thread 46 for connection and screw engagement with the ejection cannula 53 which has been inserted into and secured to the patient's aorta.

The cylindrical pump chamber section 47 formed with the pump chamber 22 of the J-shape cylindrical member 21 is made of polycarbonate resin or the like by squeeze molding and is bent to a J-shape at its inflow side. After the both ends of the pump chamber section 47 are heated to a softening temperature, the valve sections 29, 39 for the inflow and outflow end members 27, 37 are press-fit respectively into the softened ends of the bent and straight sections of the pump chamber section 47, with the inner diameters of the bent and straight end sections being expanded to meet the outer diameters of the valve sections 29, 39. After the press-fitting, the ends of the pump chamber section 47 are inwardly curved by being pressed on spherical outer surfaces of juncture portions between the valve sections 29, 39 and the coupling sections 28, 38. Accordingly, the inflow and outflow end members 27, 37 are bodily secured to the both ends of the pump chamber section 47, respectively. A suitable material for anti-thrombus is coated on those portions that get in touch with blood, such as inner surfaces of the inflow hole 23, the outflow hole 24, the valve chambers 30, 40 and the pump chamber 22 and outer surfaces of the ball valves 31, 41.

The balloon 17 of the balloon catheter 15 is inserted into the pump chamber 22 through an insertion hole 49 which opens at a boss portion 48 formed at the bent portion of the pump chamber section 47. By threadedly tightening an annular seal member 50, which the catheter 16 passes through, on the boss portion 48 through a gasket 51, the catheter 16 fluid-tightly passes through the wall of the J-shape cylindrical member 21 to come out of the pump chamber 22 and is coupled to the IABP drive unit 10. The inner diameter of the pump chamber 22 is made the same to or slightly larger than the outer diameter of the balloon 17 being expanded. The length of the pump chamber 22 is made slightly longer than that of the balloon 17. Owing to these sizes so chosen, the volume of blood which dwells within the pump chamber 22 is reduced whereby the occurrence of thrombus can be prevented. Further, since the size of the pump chamber 22 is made to be approximately same to the size of the balloon 17, it can be realized that while being pulsated, the balloon 17 is prevented from jumping and vibrating within the pump chamber 22.

A circulation assist method utilizing the balloon of the IABR system according to the present invention will be described together with the operation of the foregoing circulation assist device. The suction cannula 52 which has been inserted into and secured to the left atrium of a patient's heart is coupled to the inflow hole 23 provided on the J-shape cylindrical member 21, while the ejection cannula 53 which has been inserted into and secured to the patient's aorta is coupled to the outflow hole 24. The electrode 18 for taking an electrocardiogram is adhered to a suitable portion of the patient's body and is connected to the monitor 11 of the IABP drive unit 10. The trigger circuit 12 selects the R-wave of the electrocardiogram (electrocardiogram trigger) as trigger signals and contracts and expands the balloon 17 at such timings that have a suitable delay from the trigger signals.

Where the IABP system is employed as a circulation assist device for assisting the volume of blood stream, the balloon 17 is contracted for a contraction period of the heart cycle as the helium gas within the balloon 17 is discharged through the catheter 16 due to the negative pressure generated by the positive/negative pressure generator 13 of the IABP drive unit 10. Thus, the pump chamber 22 increases its volume thereby to generate a negative pressure. This causes the ball valve 31 to open the valve seat 32, whereby the blood streams from the left atrium into the pump chamber 22 through the suction cannula 52, the inflow hole 23 and the inflow valve 25. During this period, the ball valve 41 of the outflow valve 26 remains seated on the valve seat 42, so that the backward stream of the blood from the outflow hole 24 toward the pump chamber 22 does not take place.

For an expansion period of the heart cycle, on the other hand, the balloon 17 is expanded as being supplied with the helium gas from the gas circuit 14 of the IABP drive unit 10 through the catheter 16. Thus, the pump chamber 22 reduces its volume to increase the pressure. This causes the ball valve 41 to open the valve seat 42, whereby the blood is ejected into the patient's aorta through the valve chamber 40, the outflow hole 24 and the ejection cannula 53. During this period, the ball valve 31 remains seated on the valve seat 32, so that the backward stream of blood from the pump chamber 22 into the inflow hole 23 does not take place. By contracting and expanding the balloon 17 having a capacity of 40 milliliters, a stream volume of 3.5 litters could be obtained with the peripheral pressure of a 50 mmHg and with the pulsation number of 70-90 BPM. Further, the effectiveness was confirmed through an experiment which used an experiment dog to measure the stream volume in the left heart bypass.

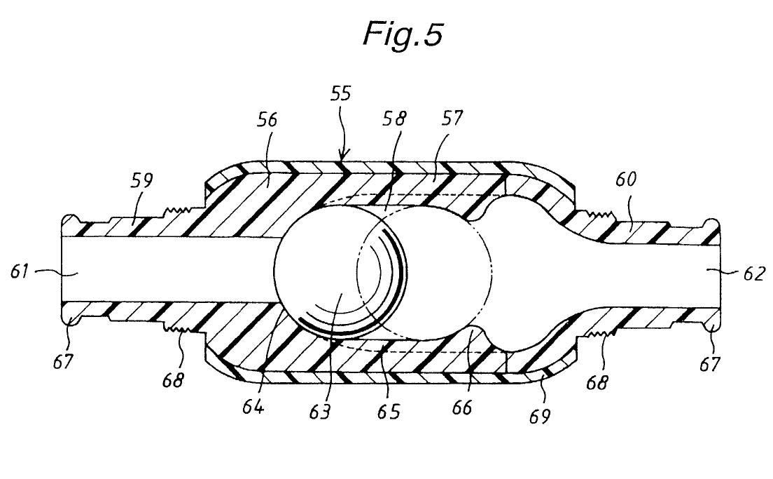

Although in the aforementioned embodiment, the inflow and outflow end members 27, 37 bodily provided at the both ends of the J-shape cylindrical member 21 incorporate the inflow valve 25 and the outflow valve 26 therein respectively, a blood stream control valve 55 shown in FIG. 5 can be used in place of any of the inflow valve 25 and the outflow valve 26. In this modified embodiment, a blood stream control valve 55 constituted independently of the cylindrical member 21 is coupled to the inflow hole 23 of the inflow end member 27 and another blood stream control vale 55 is coupled to the outflow hole 24 of the outflow end member 37. A valve chamber 58 is formed in a valve center portion 57 of a valve chamber member 56 of the blood stream control valve 55, and an inlet hole 61 and an outlet hole 62 both in communication with the valve chamber 58 are co-axially formed respectively at coupling sections 59, 60 protruding in opposite directions. A ball valve 63 contained in the valve chamber 58 is seated at an adjoining or juncture portion between the inlet hole 61 and the valve chamber 58, thereby to form a valve seat 64 which prevents the backward stream of blood. For smooth stream of blood, the valve chamber 58 gently increases its diameter as it goes away from the valve seat 64.

Like the inflow valve 25, at the inner surface of the valve chamber 58, guide protrusions 65 of three or four streams whose top portions are semicircular in cross-section extend in the direction of blood stream so as to movably guide the ball valve 63 which is leaving the valve seat 64, in the direction of blood stream. Each of the guide protrusions 65 has a sufficient height from the bottom or inner surface of the valve chamber 58, so that a larger cross-section area for blood stream than the cross-section area of each of the inflow and outflow holes 61, 62 is secured between the ball valve 63 being away from the valve seat 64 and the bottom surface of the valve chamber 58. Each protrusion 65 radially inwardly rises at its end close to the outflow hole 62 thereby to provide a stop portion 66 which confines the end in movement of the ball valve 63 toward the outflow hole 62. Each stop portion 66 takes the shape of a streamline in the direction of blood stream, and the top of each stop portion 66 is independent of, or separated from, that of any other stop portions 66, so that the blood stream within the blood stream control valve 55 is smooth and that there occur little hemolysis and little thrombus.

The valve chamber member 56 is made of polycarbonate resin or the like by injection molding to form the inlet coupling section 59 and the valve section 57 bodily but the outlet coupling section 60 being separated. The ball valve 63 made of solid silicon resin or the like is inserted into the valve chamber 58 from the side of the stop portions 66 by making slight elastic deformations of the stop portions 66 as well as the ball valve 63. Thereafter, the valve section 57 and the coupling section 60 are joined at their end surfaces by melting joint or with a bonding agent of hot-melt type. The outer surface of each of the coupling sections 59, 60 is formed with an inserting portion 67 and a male thread 68 for connection and screw engagement with a cannula, the circulation assist device or the like. A numeral 69 designates a reinforcing member made of polycarbonate resin or the like. This member 69, after heated to a softening temperature, is press-fitted over the large diameter portions of the valve section 57 and the coupling sections 59, 60. After press-fitting, the opposite ends of the reinforcing member 69 are inwardly curved along the spherical portions at respective junctures between the valve section 57 and the coupling sections 59, 60. In particular, the reinforcing member 69 reinforces the juncture of the coupling portion 60 with the valve portion 57.

In the circulation assist device which utilizes the balloon of the IABP system according to the modified embodiment of the present invention, a pair of the blood stream control valves 55 are used as being connected as follows. That is, the suction cannula 52 which has been inserted into and secured to the left atrium of the patient's heart is connected to the inlet hole 61 of one of the blood stream control valves 55, and the outlet hole 62 of the one valve 55 is connected with the inflow side of the pump chamber 22. Further, the outflow side of the pump chamber 22 is connected with the inlet hole 61 of the other blood stream control valve 55, and the ejection cannula 53 which has been inserted into and secured to the aorta is connected to the outlet hole 62 of the other stream control valve 55.

Prosthetic heart valve has been developed to replace the tricuspid, the pulmonary artery valve, the mitral valve and the aortic valve of the human heart. In fact, the prosthetic heart valve is intended to be transplanted in the human body, and thus, is made not only to prevent hemolysis and thrombosis but also to be so tough as to be durable for to-and-fro motions through several ten-millions, so that it cannot be help being expensive as a matter of course. However, it is too much quality and hence, unnecessary to use the prosthetic heart valve which is expensive and capable of transplanted into human body, as a circulation assist means which only needs to assist the blood stream for such a temporal period of time as long as twelve hours or at the most, twenty-four hours or so during a surgical operation for serious heart failure.

To the contrary, the inflow valve 25, the outflow valve 26 and the blood stream control valve 55 incorporate many features and advantages by being constructed as noted below. That is, the valve chambers 30, 40, 58 are provided in the form of monocoque in the valve sections 29, 39, 57 of the inflow end member 27, the outflow end member 37 and the valve chamber member 56, respectively. The guide protrusions 33, 43, 65 of three or four streams extend at the inner surfaces of the valve chambers 30, 40, 58 for guiding the ball valves 31, 41, 63, respectively. The ball valves 31, 41, 63 are seated on or moved away from the valve seats 32, 42, 64 formed in the valve chambers 30, 40, 58, respectively. Further, the guide protrusions 33, 43, 65 are formed smoothly, and the stop portions 34, 44, 66 are raised at one ends of the guide protrusions 33, 43, 65 for confining the ends in movement of the ball valves 31, 41,63. The stop portions 34, 44, 66 are each separated at their top portions from others of the same valve chamber. With these features, the inflow valve 25, the outflow valve 26 and the blood stream control valve 55 are capable of streaming blood smoothly within the valve chambers 30, 40, 58 thereby to reduce hemolysis and thrombosis to the minimum, are simple in construction and are low-priced, thereby being the most suitable to those valves for circulation assist means.

Although in the above-described embodiments, the pump chamber section 47 is bent at its one end to form the J-shape cylindrical body 21, it may otherwise be bent at its both ends to form a U-shape cylindrical member.

Further, it is to be noted that the blood stream control valve 55 can be generally used as those which control one-way stream of blood, in addition to being used as the foregoing circulation assist device utilizing the balloon of the IABP system.

Obviously, numerous modifications and variations of the present invention are possible in light of the above teachings. It is therefore to be understood that within the scope of the appended claims, the present invention may be practiced otherwise than as specifically described herein.

What is claimed to be secured by the Letters Patent of the United States is:

1. A circulation assist method utilizing a balloon of an IABP system, comprising the steps of: making one end and the other end of a generally cylindrical pump chamber communicate respectively with an inflow hole and an outflow hole; providing an inflow valve for permitting blood to stream from said inflow hole into said pump chamber but for preventing the backward stream of blood; providing an outflow valve for permitting blood to stream from said pump chamber to said outflow hole but for preventing the backward stream of blood; connecting said inflow hole to a suction cannula which is inserted into and secured to the left atrium of a patient's heart; connecting said outflow hole to an ejection cannula which is inserted into and secured to the patient's aorta, thereby bypassing the left ventricle; fluid-tightly incorporating into said pump chamber a balloon portion of a balloon catheter connected to said IABP drive unit; connecting said balloon catheter to said IABP drive unit; and contracting and expanding said balloon with the timings determined based on trigger signals which a trigger circuit of said IABP drive unit selects from living body signals of said patient so as to obtain one-way stream of blood.

2. A circulation assist device utilizing a balloon of an IABP system, comprising a generally cylindrical pump chamber with one and the other ends thereof connected respectively to an inflow hole and an outflow hole, an inflow valve for permitting blood to stream from said inflow hole into said pump chamber but for preventing the backward stream of blood, and an outflow valve for permitting blood to stream from said pump chamber to sad outflow hole but for preventing the backward stream of blood, and wherein a balloon portion of a balloon catheter connected to an IABP drive unit is fluid-tightly incorporated into said pump chamber and is pulsated.

3. A circulation assist device as set forth in claim 2, wherein a J-shape cylindrical member is provided with said pump chamber at a straight section thereof and with said inflow hole and said outflow hole respectively at one and the other ends thereof, and wherein said balloon portion of said balloon catheter is fluid-tightly incorporated into said pump chamber from a bent portion of said J-shape cylindrical member.

4. A circulation assist device as set forth in claims 2 or 3, wherein the inner diameter of said pump chamber is the same to or slightly larger than the outer diameter of said balloon being expanded, and wherein the length of said pump chamber is slightly longer than that of said balloon.

5. A circulation assist device as set forth in any one of claims 2 to 4, wherein:
said inflow valve is formed with guide protrusions of three or four streams which extend in the direction of blood stream for guiding a ball valve within a valve chamber communicating with said pump chamber at the side of said inflow hole; a valve seat on which said ball valve is seated at the side of said inflow hole of said valve chamber is formed for preventing the backward stream from said pump chamber toward sad inflow hole; said guide protrusions are raised at the side of said pump chamber thereby to confine the movable end of said ball valve; said outflow valve is formed with guide protrusions of three or four streams which extend in the direction of blood stream for guiding a ball valve in a valve chamber communicating with an outflow side of said pump chamber; a valve seat on which said ball valve is seated at the side of said valve chamber close to said pump chamber is formed for preventing the backward stream from said outflow hole toward said pump chamber; and said guide protrusions are raised at the side of said outflow hole thereby to confine the movable end of said ball valve.

6. A blood stream control valve wherein: a valve chamber is formed within a valve chamber member with both ends thereof being communicating respectively with an inlet hole and an outlet hole;
guide protrusions of three or four streams for movably guiding a ball valve extend in the axial direction thereof; a valve seat on which a ball valve is seated for preventing the backward stream of blood from said outlet hole toward said inlet hole is formed at the side close to said inlet hole, of said valve chamber; and a stop portion for confining the movable end of said ball valve when blood makes the ball valve open the valve seat thereby to stream from said inlet hole to said outlet hole is formed by raising the ends at the side of said outflow hole, of said guide protrusions.

7. A circulation assist device used in cooperation with a widespread IABP system, comprising a generally cylindrical member formed with a pump chamber at its mid portion; an inflow valve provided at the upstream side of said pump chamber for permitting blood to stream in one-way only toward said pump chamber; an outflow valve provided at the downstream side of said pump chamber for permitting blood to stream in one-way only from said pump chamber toward said outflow valve; and wherein said cylindrical member is bent at a portion close to its one end to provide a hole for fluid-tightly incorporating a balloon of said IABP system so that the contraction and expansion of said balloon within said pump chamber is utilized as power source to suck blood into said pump chamber and then eject blood from said pump chamber.
